(19) United States
(12) Patent Application Publication
(10) Pub. No.: US 2014/0010793 A1
(43) Pub. Date: Jan. 9, 2014

(54) Method for Treating Cancers with Dendritic Killer Cells and Pharmaceutical Composition

(71) Applicant: FullHope Biomedical Co., Ltd., Taipei City (TW)

(72) Inventor: Jan-Mou LEE, Taipei City (TW)

(21) Appl. No.: 13/918,736

(22) Filed: Jun. 14, 2013

(30) Foreign Application Priority Data
Jul. 5, 2012 (TW) 101124291

(51) Int. Cl. C12N 5/0784

(52) U.S. Cl. CPC C12N 5/0639
USPC 424/93.71; 435/325

(57) ABSTRACT

The present invention discloses a use of dendritic killer cell population for manufacturing medication. The dendritic killer cell population is generated by culturing peripheral blood mononuclear cells with effective amounts of various cytokines for an appropriate time period, and the conserved cytokine is IL-15. Meanwhile a pharmaceutical composition comprises the above dendritic killer cell population for treating cancers is also disclosed in the present invention.

### CROSS-REFERENCE TO RELATED APPLICATIONS

This Non-provisional application claims priority under 35 U.S.C. §119(a) on Patent Application No(s). 101124291 filed in Taiwan, Republic of China, 07, 05, 2012, the entire contents of which are hereby incorporated by reference.

### FIELD OF THE INVENTION

The present invention relates to a method for treating cancers and pharmaceutical composition comprising the same, especially relates to a method of treating cancers with dendritic killer cell population generated after culturing by cytokines, and further, the pharmaceutical composition comprises the above dendritic killer cell population.

### BACKGROUND OF THE INVENTION

Human body will recognize the extraneous matter and start a series of defending process. This defense system is named as immune system. There are many different cells such as leukocytes and lymphocyte, and different protein factors such as immunoglobulins and cytokines working coordinately to protect the body. The immune systems are traditionally divided into innate and adaptive immune systems. Innate immune system is including soluble complement system, polymorphonuclear neutrophils, macrophages and natural killer cells. Adaptive immune system is including humoral and cellular immunity. Humoral immunity as well as cellular immunity involves lymphocyte, lymphokine and immunological memory system. The long-lasting immune memory mounts quick and strong immune responses towards the same pathogen which has invaded the body.

Immune system may respond to different pathogens due to the diversity of major histocompatibility complex (MHC) molecules. The endogenous and exogenous antigens derived from pathogens, are assembled with MHC molecules on the surface of antigen-presenting cells (APC) and then presented to T cells expressing corresponding T cell receptors. MHC in the human beings can be called Human Leukocyte Antigen, HLA, which can be categorized into class I, class II, and class III. HLA class I is widely expressed on all the somatic cells but Class II distribution is restricted to macrophages, B cells and dendritic cells.

Dendritic cells (DC), which have the broadest range of antigen presentation, are professional APC, and named by the appearance of dendrites extending from the cell body. DCs reside in the periphery of body as immature DCs (imDCs). Once pathogen invades human bodies, imDCs capture pathogen-derived antigens, migrate to draining lymph nodes to become mature DCs (mDCs), and present antigens to corresponding T cells there. Therefore, dendritic cells are the starter of the pathogen-specific cellular immune responses.

Natural killer (NK) cells, a key player of innate immune system, spontaneously kill tumor or virally infected cells prior to activation. Mechanisms underlying cytotoxicity of NK cells are grouped into two parts: a) interaction of cell surface tumor necrosis factor superfamily members and their receptors which leads to apoptosis of target cells, (b) release of soluble perforin and granzymes. NK cells are rich with small granules in their cytoplasm contain special proteins such as perforin and proteases known as granzymes. Upon release in close proximity to a cell slated for killing, perforin forms pores in the cell membrane of the target cell through which the granzymes and associated molecules can diffuse in, leading to destruction of target cells. Once virally infected cells or tumor cells have been killed, viral genomic content (CpG or poly I:C), cellular metabolites, and bystander cytokines such as IFN-•, IL-12 and TNF-• would further activate and augment NK cell activity in term of cytotoxicity and effector cytokine production. Therefore NK cells serve as key innate effector cells targeting to virally infected cells and tumor cells in a non-antigen specific manner while DCs in adaptive immune system trigger antigen-specific cytotoxic T cells which can further clear the infection. Patients deficient in NK cells are proved to be highly susceptible to early phases of herpes virus infection.

Interferon-producing killer dendritic cells (IKDCs), a recently identified leukocyte population in mice, express phenotypes of non-T (CD3⁻), non-B (CD19⁻), intermediate levels of CD11c, and high levels of B220 and NK-specific markers, including NK1.1, DX5, NKG2D and Ly49 family receptors. IKDCs functionally resemble NK cells in cytotoxicity against tumor cells and in production of abundant IFN-•. On the other hand, upon stimulation with CpG or tumor cells, IKDCs down-regulate NKG2D, up-regulate MHC II, and acquire moderate APC-like activity that activates antigen-specific T cells. Despite acquisition of APC activity after certain stimulations, IKDCs appear to belong to the NK lineage rather than DC lineage. IKDCs express NK-specific Ncr-1 transcripts (encoding NKp46) but not PU.1 that is predominantly expressed in DCs and plasmacytoid DCs. Furthermore, IKDC development parallels NK cells in their strict dependence on the IL-15 cytokine system. Therefore, the putative IKDCs are functionally and developmentally similar to NK cells. Although debates regarding tumoricidal activity and cell lineage development of IKDC were raised herein, further investigations were limited by rare abundance of IKDC in periphery. The frequency of IKDCs in a mouse spleen is below 0.01%, and is even lower in the lymph nodes. Therefore, cumbersome procedure is required for the purification of IKDCs, and the yield is low. This problem has limited the use of IKDCs in research and in application.

### SUMMARY OF THE INVENTION

According to the abovementioned disadvantages of the prior art, Applicant put a lot of efforts in the past years and successfully screens out cells which have the functions of both natural killer cells and dendritic Cells. The abovementioned cells are defined as Dendritic killer cell (hereafter called DKC), also be called cytotoxic dendritic cell (cytoDC). However, it is noted that the DKC constitutes less than 0.01% of peripheral lymphocytes.

Therefore, Applicant successfully makes trace DKC of human blood increase in an amount of 200-fold to 400-fold, and further use the toxicity of DKC to kill tumor cells and treat cancers. According to the abovementioned, the present invention provides a method of treating cancers with DKC and pharmaceutical composition comprising the same. Moreover, the abovementioned DKC are generated by culturing with cytokines.

The present invention provides a method for treating cancers, and the method comprises the following steps: first, DKCs are obtained from a cancer patient. And then, a DKC population is generated by culturing DKCs. Finally, the DKC population will be delivered into the cancer patient. Preferably, the DKC population is delivered into the cancer patient through intravenous injection.

Preferably, the DKC population is generated by culturing with cytokines according to the following steps. First, a step of obtaining a peripheral blood mononuclear cell population from human blood is performed. Then, at least one of the cytokines with an effective amount is added to mix with the peripheral blood mononuclear cell population and placed for an appropriate period. Finally, the DKC population will be sorted. The abovementioned cytokines comprise IL-15. Preferably, the abovementioned cytokines further comprise IL-12.

The present invention further provides a pharmaceutical composition containing a plurality of human DKCs, wherein the DKCs are HLA-G⁻CD14⁻CD19⁻CD3⁻CD56⁺HLA-DR⁺.

The present invention further provides a use of DKC population for manufacturing medication. Preferably, the medication is administered to the cancer patient to inhibit tumor growth. Preferably, the abovementioned DKC population can form a pharmaceutical composition with a buffer.

The present invention further provides a pharmaceutical composition for treating cancers, and the pharmaceutical composition comprises an effective medical dosage of DKC population and a buffer. Preferably, the pharmaceutical composition is administered to the cancer patient to inhibit the tumor growth.

Preferably, the DKC population comprises surface markers of CD14⁻HLA-G⁻CD3⁻CD19⁻HLA-DR⁺CD56⁺.

Preferably, the buffer can be selected from a group consisting of phosphate buffer and saline solution.

Preferably, the pharmaceutical composition comprises DKC population which is generated ex vivo by culturing the DKC obtained from human blood with effective amounts of various cytokines. Preferably, the human blood is collected from a cancer patient. Preferably, the abovementioned cytokine comprises IL-15. Preferably, the abovementioned cytokine further comprises IL-12.

Preferably, the abovementioned pharmaceutical composition can be administered to a cancer patient to inhibit the tumor growth. Preferably, the abovementioned pharmaceutical composition is administered through injection.

The present invention further provides a method of treating a tumor in a human subject in need thereof; the method comprises to administer the abovementioned composition to the human subject.

The features and advantages of the present invention will be understood and illustrated in the following specification and FIGS. 1˜8B.

### BRIEF DESCRIPTION OF THE DRAWINGS

FIG. 1 is flow diagram showing a method according to an embodiment of the present invention for cultivating DKC population;

FIG. 2A to FIG. 2C are diagrams showing the results of detecting and screening cultivated DKC population by a flow cytometer;

FIG. 3A to FIG. 3C are diagrams showing the results of detecting cell survival by a flow cytometer after reacting DKC population with tumor cells;

FIG. 4A to FIG. 4B are diagrams showing the results before and after addition of tumor cells into the pharmaceutical composition according to an embodiment of the present invention;

FIG. 5 is a diagram showing the cell mortality rate of control and pharmaceutical composition according to an embodiment of the present invention;

FIG. 6A to FIG. 6B are diagrams showing the results before and after addition of ovarian cancer cells into the pharmaceutical composition according to an embodiment of the present invention;

FIG. 7A to FIG. 7B are diagrams showing the antigen presenting results of cultured DKC detecting by a flow cytometer; and

FIG. 8A to FIG. 8B are diagrams showing the results of γ-interferon presenting by activated T cells detecting by a flow cytometer.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

As used herein, the term “Dendritic killer cells” or “DKC” is intended to refer to the cells with both cytotoxicity and antigen presenting cell (APC) activity.

As used herein, the symbol “+” means that the cell surface marker expresses on the surface of the cells and has a larger expressed amount measured by flow cytometer than that of the negative control.

As used herein, the symbol “−” means that the cell surface marker does not express on the surface of the cells and has an expressed amount equal to that of the negative control.

Preferably, all abovementioned expressed amount of the cell surface markers are measured by flow cytometer, however, the present invention is not limited thereto.

As used herein, the term “Interleukin” means a group of cytokines that were first seen to be expressed by white blood cells (leukocytes). It has since been found that interleukins are produced by a wide variety of body cells. The function of the immune system depends in a large part on interleukins.

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention. To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Preferably, several sorting or screening steps are performed by a flow cytometer, and a target cell population will be screened out by utilizing at least one flow cytometer to identify different surface markers of different cells. Flow cytometry allows for single cell analysis at speeds far surpassing any other single cell analysis technology in the art. This enables a statistically significant number of cells to be analyzed faster than using other alternative techniques. In a preferred embodiment, a flow cytometer is used with any suitable sample preparation robot or liquid handler that is known in the art. Furthermore, a single laser flow cytometer is used in an embodiment for the analyzing step. In another embodiment, a multi-laser flow cytometer is used for the analyzing step and the present invention is not limited thereto.

At first Applicant put a lot of efforts in the past years and successfully screens out cells which have the functions of both natural killer cells and dendritic Cells. These cells are defined as dendritic killer cell (hereafter called DKC) as mentioned above and have surface markers of HLA-G⁻CD14⁻CD19⁻CD3⁻CD56⁻HLA-DR⁺.

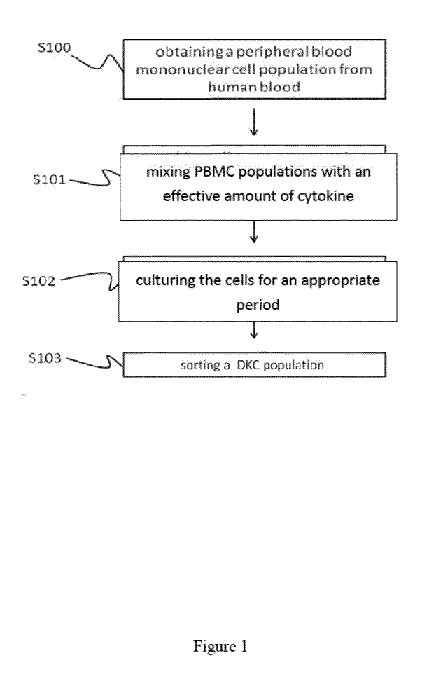

As abovementioned, the DKCs are identified from human peripheral blood. In the following, the method disclosed in the present invention of cultivating the DKCs from human peripheral blood for further use as DKC population in pharmaceutical composition will be illustrated through FIG. 1.

Please refer to FIG. 1, step S100 of obtaining a peripheral blood mononuclear cell population from human blood is performed at first. And then, step S101 of adding an effective amount of at least a cytokine to mix with the peripheral blood mononuclear cell population is performed. Preferably, the cytokine comprises an effective amount of Interleukin-15 (hereafter “IL-15”). The following step S102 is to place the peripheral blood mononuclear cell population for an appropriate period. Finally, a DKC population will be sorted in step S103.

Preferably, the abovementioned cytokine further comprises Interleukin-12 (hereafter “IL-12”). Preferably, the concentration of abovementioned IL-15 is 10 ng/mL, and the concentration of IL-12 has a value between 0.5˜20 ng/mL.

Preferably, the abovementioned step S100 further comprises the following steps. At first, the human blood of 40 ml is collected and the human peripheral blood mononuclear cell (hereafter “PBMC”) is sorted. Then T cells and B cells are removed from the peripheral blood mononuclear cell population. The human peripheral blood mononuclear cells comprise the following five categories of cells: monocytic cells, small cells, lymphoid cells, large cells and large and granular cells. Flow cytometry can be first used to select one or more types of cells for follow steps. Preferably, the cell comprises monocytic cells or lymphoid cells or both, but the present invention is not limited thereto.

Preferably, the abovementioned appropriate period means that IL-15 and the peripheral blood mononuclear cell population are both put into a media for a period to let cell proliferation process. Preferably, the appropriate period is the seventh day after starting the abovementioned cultivating step.

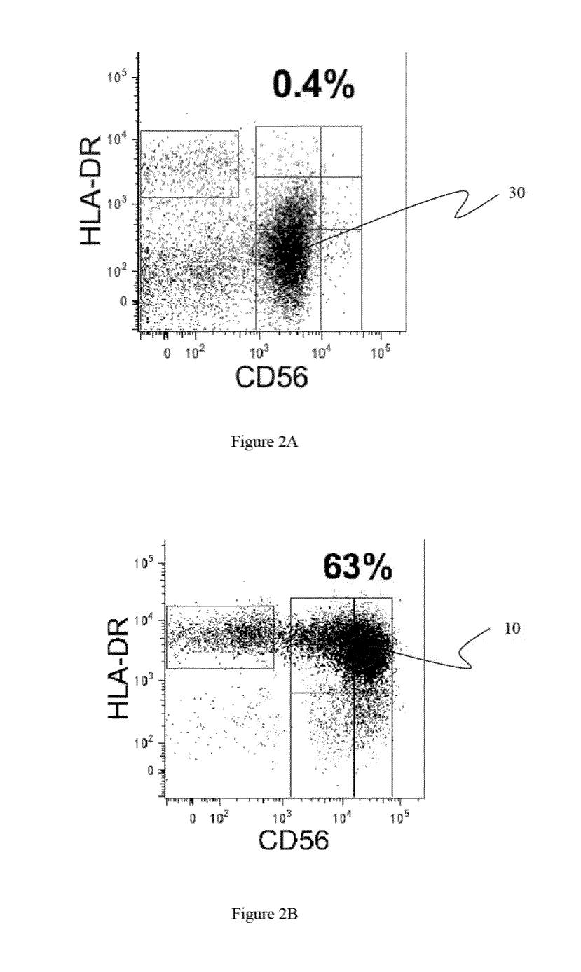

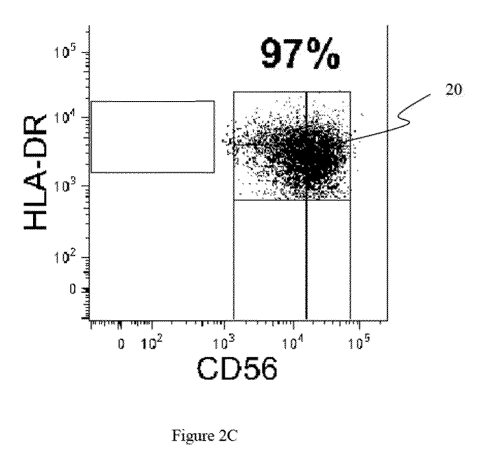

Please refer to FIG. 2; FIG. 2A illustrates the results of detecting the surface markers of CD56 and HLA-DR by a flow cytometer after removing the T cell and B cell (CD3⁻CD19⁻PBMC) from human peripheral blood mononuclear cells and before cultivating. FIG. 2B illustrates the results of detecting the surface markers of CD56 and HLA-DR by a flow cytometer at the seventh day after starting the cultivating step. FIG. 2C illustrates the results of sorting DKC population by a flow cytometer.

As shown in FIG. 2A, the counts of the cells which have natural killer cell surface marker (CD56⁺) and dendritic Cell surface marker (HLA-DR⁺) are much fewer. And further, the cells 30 positioned at the central portion are natural killer cells which have the surface marker of CD 56 but not HLA-DR. Please refer to FIG. 2B, the cells will transfer to the DKC 10 which has both natural killer cell surface marker (CD56⁺) and dendritic cell surface marker (HLA-DR⁺). The DKCs expand the counts and further let natural killer cells transfer to DKC. Finally, FIG. 2C illustrates the sorted cells selected by flow cytometer, the DKC population 20 which has the surface marker of HLA-G⁻CD14⁻CD 19⁻CD3⁻CD56⁺HLA-DR⁺.

It is noted that the abovementioned appropriate period is the preferred embodiment; however, the present invention is not limited thereto. That is, the step S103 can be performed on the fourth day after cultivating or on the tenth day after cultivating. Furthermore, the steps S101˜S103 can be repeatedly performed after the step S103. That is, no-adherent cells will be collected again and the counts of the dendritic killer cells will be expanded to an expect value by repeating the abovementioned steps.

Preferably, the method disclosed in the present invention is processed ex vivo, wherein the human blood is collected from a cancer patient. And further, a cancer, which the cancer patient suffers from, can be selected from a group consisting of squamous cell carcinoma, lobular carcinoma in situ, liver cancer, nasopharyngeal carcinoma, lung cancer, bone cancer, pancreatic cancer, skin cancer, head and neck cancers, malignant melanoma, cervical cancer, ovarian cancer, colon cancer, anal cancer, stomach cancer, breast cancer, testicular cancer, fallopian tube cancer, endometrial cancer, cervical cancer, vaginal cancer, vulvar cancer, non-Hodgkin lymphoma, Hodgkin lymphoma, esophageal cancer, thyroid cancer, adrenal cancer, cancers of mesothelial and soft tissue, urethra cancer, cancer of penis, prostate cancer, acute leukemia, chronic leukemia, lymphomas, bladder cancer, ureteral cancer, renal cell carcinoma, urothelial carcinoma, cancer of central nervous system, primary central nervous system lymphoma, glioma, pituitary tumor, Kaposi's sarcoma, squamous cell cancer and their metastasis.

According to abovementioned, the DKC population 20 sorted from cultivating trace amount of DKC in human blood increase in an amount of 200-fold to 400-fold. Therefore, the cultivating DKC population can be administered to the cancer patient as medication to inhibit tumor growth. The DKC population 20 is further used to manufacture medication for treating cancers, that is, the DKC population 20 can form a pharmaceutical composition with buffer to effectively apply for cancer. Preferably, the abovementioned concentration for DKC population is 10⁶ cells/mL.

Preferably, the present invention further provides a pharmaceutical composition for treating cancer, and the pharmaceutical composition further comprises an effective medical dosage of DKC population generated by culturing with cytokines and a buffer. Preferably, the DKC population comprises the surface markers of CD14⁻HLA-G⁻CD3⁻CD19⁻HLA-DR⁺CD56⁺.

Preferably, the abovementioned DKC population is generated ex vivo by culturing the DKC obtained from peripheral blood of the cancer patient and the pharmaceutical composition is administered to a cancer patient. That is, the DKC took from the cancer patient is manufactured to the pharmaceutical composition through cultivating, and administered back to the cancer patient to inhibit tumor growth as medication.

Preferably, the pharmaceutical composition is transferred through injection, but the present invention is not limited thereto.

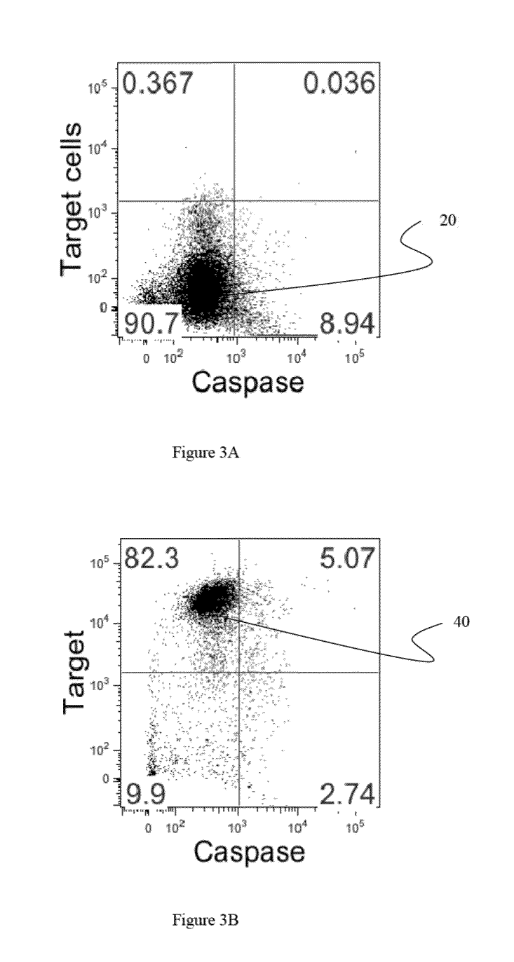

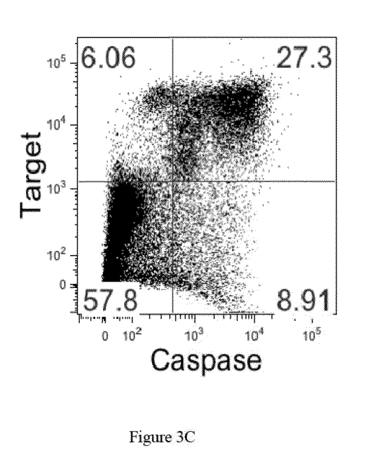

In order to prove the DKC population can be used for manufacturing medication and the abovementioned medication is used for treating cancers, Applicant reacts the cultivating DKC population 20 with target cell (K562) 40 and measures the tumor cell death by a flow cytometer. As shown in FIG. 3A to FIG. 3C, the vertical axis represents the sizes of the cells and the transverse axis represents the content of Caspase 6 in the cells. It is noted that Caspase 6 is an important protease in apoptosis so that the cell is dead or dying if Caspase 6 of the cell is dyed. That is, the killing efficiency of the DKC can be detected by detecting the content of Caspase 6 in the target cells.

Please continue referring to FIG. 3A to FIG. 3C, each of the figures is divided into four blocks: the upper portion represents the tumor cells, the lower portion represents the DKC, the left portion represents the living cells and the right portion represents the dead cells. Preferably, the target cells are K562 cells. As shown in the figures, FIG. 3A shows the abovementioned cultivating DKC 20. FIG. 3B shows the tumor cell 40 before reacting with any other cells, therefore, all tumor cells distribute at the upper left portion and no dyed Caspase 6 existed meaning the tumor cells are all living cells.

Please refer to FIG. 3C, FIG. 3C shows the result of cultivating DKC population with target tumor cells for 40 min. After reacting the tumor cells with the DKC population, the cells distributed at the upper portion apparently shift to right showing about 85% of cells are dyed with caspase 6. That is, a lot of the tumor cells decease. However, most DKC population at the lower portion survived showing a great toxicity of DKC to tumor cells.

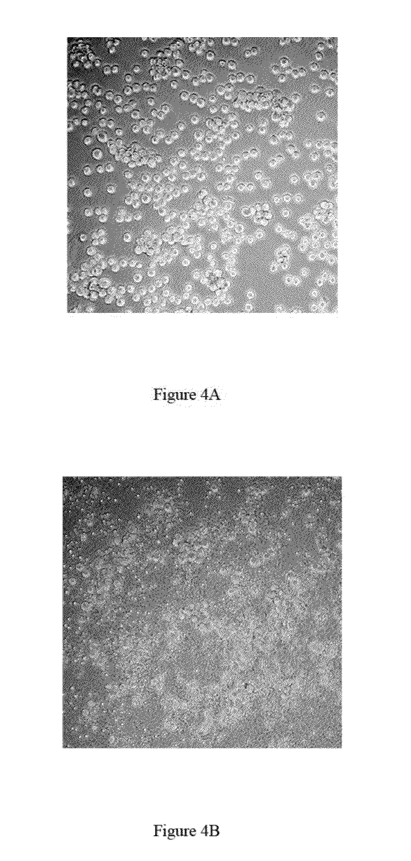

Please refer to FIG. 4A to FIG. 4B, they show the results of cell growth for original tumor cells and tumor cells cultivating with DKC population for 40 min. As shown in FIG. 4A, the tumor cells grow perfectly in media before adding DKC population. However, a lot of the tumor cells decease after reacting with the DKC as shown in FIG. 4B.

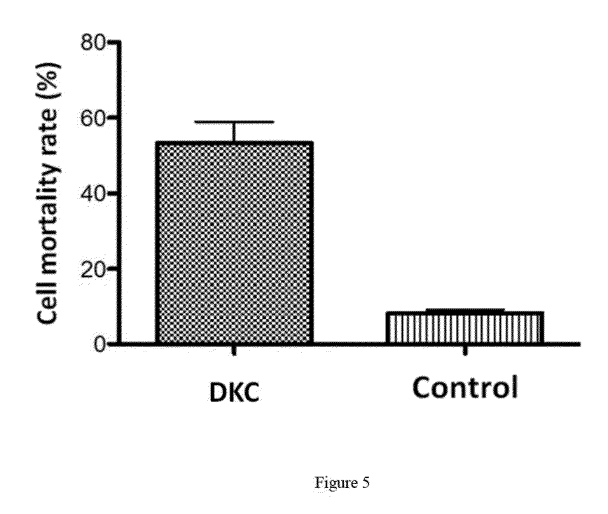

Please refer to FIG. 5, it shows the cell mortality rate of control and pharmaceutical composition with tumor cell concentration of 10⁶ cells/mL. As shown in the figure, the tumor cells decreases over half after adding DKC population, that is, over half of the tumor cells are killed by DKC population. However, the control (media only) decreases less than 10%.

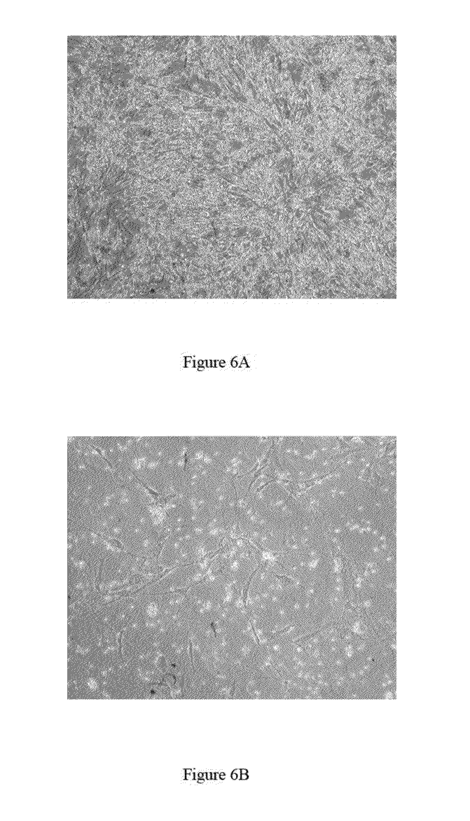

Please refer to FIG. 6A to FIG. 6B, which are the results before and after addition of 10⁶ cells/mL DKC populations into ovarian cancer cells obtained from a cancer patient. As shown in FIG. 6A, the ovarian cancer cells obtained from patient surgery were put in media and grew normally before react with DKC. And then, after reacting with DKC population cultivating from the peripheral blood of the patient with ovarian cancer for 40 min, a lot of the tumor cells decreases as shown in FIG. 6B.

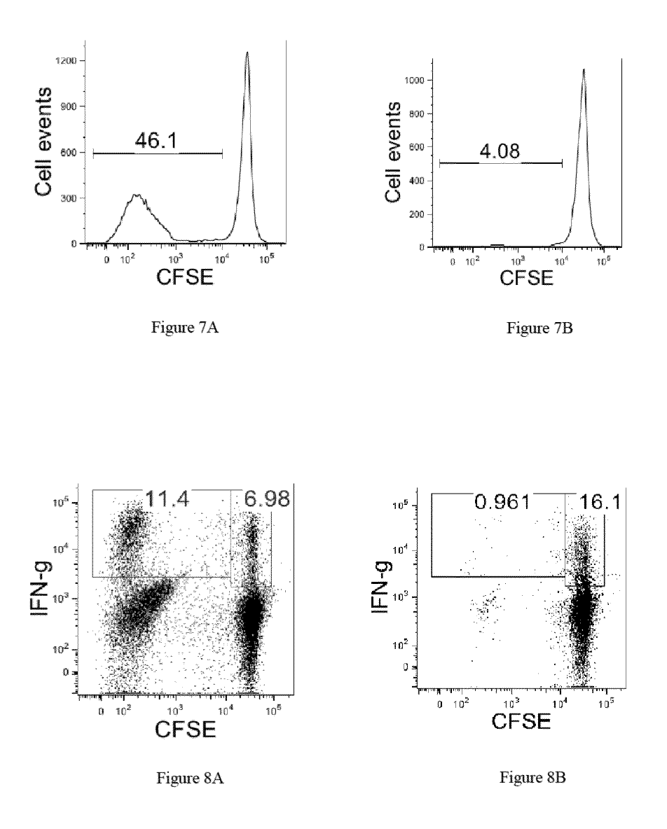

In order to prove the cell population used in the present invention was DKC, that is, containing the cytotoxicity and antigen presenting function. The abovementioned DKC shows cytotoxicity by killing tumor cells, and the below experiment proves the existence of antigen presenting function. Please refer to FIG. 7 to FIG. 8.

Applicant cultures DKC prepared from the first subject with the allogeneic T cell in peripheral blood mononuclear cells of a second subject. And then, the T cell activation and proliferation of the second subject will be measured by a flow cytometer. Please refer to FIG. 7; a mixed leukocyte reaction is utilized by culturing the DKC sorted from PBMC of the first subject with CFSE-labeled T cells of the second subject. And then, the DKC of the first subject will activate the T cells of the second subject. Therefore, the activation and proliferation of T cells of the second subject can represent allo-stimulatory APC activity of DKC of the first subject. The abovementioned CFSE is a dye for quantifying the degree of cell proliferation. After each cell proliferation, the fluorescence intensity of CFSE will decrease. However, when CFSE labeling is performed optimally, approximately 7-8 cell divisions can be identified before the CFSE fluorescence is too low to be distinguished above the autofluorescence background. Thus CFSE represents an extremely valuable fluorescent dye for immunological studies, allowing lymphocyte proliferation, migration and positioning to be simultaneously monitored.

Please refer to FIG. 7A; it shows a result of reacting the DKC cultivating from the first subject and T cell labeled with CFSE from the second subject. As shown in the figures, the vertical axis represents the content of the cells and the transverse axis represents the fluorescence intensity of CFSE within the cells. Please continue referring to FIG. 7A; 46.1% T cell of the second subject have been activated by the DKC of the first subject to processing cell proliferation. FIG. 7B is negative control. In negative control, T cell of the second subject is lonely put inside s medium, and only 4.08% of T cell has been activated and proliferate. That is, the DKC identified and screened by the present invention actually has the antigen presenting cell activity.

Please refer to FIG. 8; it shows a result of interferon-γ(hereafter “IFN-γ”) in T cells after reaction from FIG. 7. As shown in FIG. 8A, most T cells activated by DKC population contain IFN-γ (distribute at the upper left portion) and no detectable IFN-γ T cells T cell of the second subject lonely put inside medium as shown in FIG. 8B.

To sum up, DKC is a cell with function from both natural killer cell and dendritic cell. Although DKC plays an important role in immunoreactions, the content of the DKC in the human body is very rare. The trace DKC of the human blood can be expanded from 200-fold to 400-fold by the cultivating, screening and sorting technique disclosed in the present invention. Moreover, the DKC used in the present invention is medication for treating cancer. That is, the pharmaceutical composition for treating cancer comprises a dendritic killer cell population and a buffer and the pharmaceutical composition can be administered back to the same patient to treat cancer.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

1. A pharmaceutical composition for treating cancers comprising an effective medical dosage of dendritic killer cell population and a buffer fitted in with cellular acceptance, wherein the dendritic killer cell population is generated by culturing with cytokines.

2. The pharmaceutical composition according to claim 1, wherein the dendritic killer cell population comprises surface markers of HLA-G⁻CD14⁻CD19⁻CD3⁻CD56⁺HLA-DR⁺.

3. The pharmaceutical composition according to claim 1, wherein the buffer can be selected from a group consisting of phosphate buffer and saline solution.

4. The pharmaceutical composition according to claim 1, wherein the cytokines comprises an effective amount of IL-15.

5. The pharmaceutical composition according to claim 4, wherein the cytokines further comprises an effective amount of IL-12.

6. The pharmaceutical composition according to claim 1, wherein dendritic killer cell population is generated ex vivo by culturing dendritic killer cells of human blood with the cytokines.

7. The pharmaceutical composition according to claim 6, wherein the human blood is collected from a cancer patient.

8. The pharmaceutical composition according to claim 7, the pharmaceutical composition is administered to the cancer patient for inhibiting the tumor growth.

9. The pharmaceutical composition according to claim 1, the pharmaceutical composition is transferred through injection.

10. A use of dendritic killer cell population for manufacturing medication, wherein the medication is applied for treating cancer and the dendritic killer cell population is generated by culturing with at least a cytokine.

11. The use according to claim 10, wherein the medication is administered to a cancer patient for inhibiting the tumor growth.

12. The use according to claim 10, wherein the cytokine comprises an effective amount of IL-15.

13. The use according to claim 12, wherein the cytokine further comprises an effective amount of IL-12.

14. The use according to claim 10, wherein the dendritic killer cell population can form a pharmaceutical composition with a buffer.

15. A method for treating cancers comprising the following steps:
obtaining dendritic killer cells from a cancer patient;
generating a dendritic killer cell population by culturing the dendritic killer cells; and
delivering the dendritic killer cell population into the cancer patient.

16. The method according to claim 15, wherein the dendritic killer cell population is obtained by culturing the dendritic killer cells with at least a cytokine.

17. The method according to claim 16, wherein the cytokine comprises IL-15.

18. The method according to claim 17, wherein the cytokine further comprises IL-12.

19. The method according to claim 15, wherein the dendritic killer cell population is collecting from human blood of the cancer patient.

20. The method according to claim 15, wherein the dendritic killer cell population is administered to the cancer patient through intravenous injection.

21. A pharmaceutical composition containing a plurality of human DKCs, wherein the DKCs are HLA-G⁻CD14⁻CD19⁻CD3⁻CD56⁺HLA-DR⁺.

22. A method of treating a tumor in a human subject in need thereof, the method comprising administering to the human subject the composition of claim 1.

23. A method of treating a tumor in a human subject in need thereof, the method comprising administering to the human subject the composition of claim 21.
